(19) United States
(12) Patent Application Publication
(10) Pub. No.: US 2013/0150748 A1
(43) Pub. Date: Jun. 13, 2013

(54) APPARATUS FOR COMBINING DRUG EFFECT INTERACTION BETWEEN ANAESTHETICS AND ANALGESICS AND ELECTROENCEPHALOGRAM FEATURES FOR PRECISE ASSESSMENT OF THE LEVEL OF CONSCIOUSNESS DURING ANAESTHESIA

(71) Applicant: William Kai Jensen, Marstal (DK)

(72) Inventor: William Kai Jensen, Marstal (DK)

(73) Assignee: QUANTIUM MEDICAL S.L., Barcelona (ES)

(21) Appl. No.: 13/812,675

(22) PCT Filed: Jul. 18, 2011
(86) PCT No.: PCT/DK2011/000084

§ 371 Date: Jan. 28, 2013

(30) Foreign Application Priority Data
Jul. 23, 2010 (DK) PA 2010 00680

(51) Int. Cl. A61B 5/00; A61B 5/0476

(52) U.S. Cl. CPC A61B 5/4821; A61B 5/0476
USPC 600/544

(57) ABSTRACT

The present invention consists of an apparatus for the on-line identification of drug effect using drug interactions and physiologic signals, in particular the interaction between anaesthetics and analgesics combined with the electroencephalogram for precise assessment of the level of consciousness in awake, sedated and anaesthetised patients. In a preferred embodiment the apparatus comprises: two infusion devices, for example syring pumps, which are connected to the patient (1) adapted to deliver hypnotics (2) and analgesics (3). The infusion data from the pumps are fed into an interaction model (5); an interaction model characterized by a Neural Network which is adapted to estimate the parameters of the model online and in real-time for drug interaction between anaesthetics and an analgesics, an EEG instrumentation amplifier; a processing unit adapted to calculate an EEG index of the level of consciousness (ELC); a fuzzy logic reasoner adapted to merge extracted EEG parameters into an index.

### BACKGROUND OF THE INVENTION

### Introduction to Anaesthesia

In a simplistic definition, anaesthesia is a drug induced state where the patient has lost consciousness, loss of sensation of pain, i.e. analgesia, furthermore the patient may be paralysed as well. This allows the patients to undergo surgery and other procedures without the distress and pain they would otherwise experience.

One of the objectives of modern anaesthesia is to ensure adequate level of consciousness to prevent awareness without inadvertently overloading the patients with anaesthetics which might cause increased postoperative complications. The overall incidence of intraoperative awareness with recall is about 0.1-1%, but it may be much higher in certain high risk patients, like multiple trauma, caesarean section, cardiac surgery and haemodynamically unstable patients. Intraoperative awareness is a major medico-legal liability to the anaesthesiologists and can lead to postoperative psychosomatic dysfunction in the patient, and should therefore be avoided.

A method for assessing the level of consciousness during general anaesthesia is found in the Observers Assessment of Alertness and Sedation Scale (OAAS). The OAAS is a 6 level clinical scale where the levels 3 to 5 corresponds to awake while the levels 2 to 0 indicates anaesthesia where level 0 is the deepest level, the table below shows the definition of the scale.

### The OAAS Scale

| Score | Responsiveness |
|---|---|
| 5 | Responds readily to name spoken in normal tone. |
| 4 | Lethargic response to name spoken in normal tone. |
| 3 | Responds only after name is called loudly or repeatedly. |
| 2 | Responds only after mild prodding or shaking. |
| 1 | Responds only after noxious stimuli. |
| 0 | No response after noxious stimuli. |

Other clinical scales exist however the disadvantage of using clinical scales in practice is that they cannot be used continuously and that they are cumbersome to perform. This has lead to the investigation into automated assessment of the level of consciousness. The most prevailing method is the analysis of the EEG where a scalp EEG is recorded and subsequently processed by an algorithm which maps the EEG into an index typically in the 0-100 range.

The processing of the EEG often involves a spectral analysis of the EEG or more advanced signal processing methods such as Symbolic Dynamics, Entropy, Bispectral analysis or simultaneous time-frequency analysis of the EEG such as the Choi-Williams distribution and Lempel Zev complexity have been proposed as correlates to the level of consciousness. The EEG can then be classified into frequency bands where delta is the lowest activity, followed by theta, alpha and beta activity. In general, a decrease in the mean or spectral edge frequency of the EEG is occurring when the patient is anaesthetized.

Several parameters may then be combined into a single index by using a discriminatory function such as logistic regression, fuzzy logic, neural networks a.o.

The EMG is known as influencing and superimposing the EEG rendering the interpretation of the EEG difficult due to a lower signal to noise ratio. The EMG is dominant in the frequency range from 40-300 Hz but it is present in the lower frequencies down to 10 Hz as well. This means that the EEG and the EMG cannot be separated by simple band-pass filtering. Therefore other methods should be sought in order to separate these two entities, based on the assumption that some characteristics of the two are different. The complexity of the EEG and the EMG is probably different, although both signals show highly non linear properties.

### SUMMARY OF THE INVENTION

The present invention relates to a method and apparatus for assessing the level of consciousness during general anaesthesia. For this purpose a signal is recorded from the patients scalp with surface electrodes, the recorded signal is defined as:

S=EEG+EMG+artifacts,

where the EEG is the electroencephalogram, the EMG is the facial electromyogram and the artifacts are all other signal components not derived from the EEG or EMG. The artifacts are typically 50/60 Hz hum, noise from other medical devices such as diathermy or roller pumps or movement artifacts.

However, the EMG is typically the most important source of noise which interferes with the EEG. It is difficult to separate the EEG and the EMG because they have an important spectral overlap, therefore classical filtering techniques fail to separate the EMG from the EEG. The influence is apparent, the article by Messner et al. The bispectral index declines during neuromuscular block in fully awake persons. Anesth Analg. 2003 August; 97(2):488-91 shows that a level of consciousness index is significantly changed when the EMG activity is removed by the administration of a Neuro Muscular Blocking Agent (NMBA). The level of consciousness index referred to in this article is the Bispectral Index (BIS), commercialised in the BIS monitor by Aspect Medical, Ma, USA.

Other methods have been examined for assessing the complexity of the EEG such as Entropy, Limpel-Zev complexity and Bispectral analysis; also Symbolic Dynamics method has been explored to examine extract features from the EEG.

The patent application EP 1 741 388 A1 discloses a method to determine whether one drug inducing high frequency EEG was administered to a subject in general anaesthesia. It is claimed to be a method where at least one drug is a NMDA (N-Methyl-D-aspartate) antagonist and at least one drug belongs to a group including ketamine, S-ketamine, nitrous oxide, and xenon.

It contains a method for monitoring the cerebral state of a subject by obtaining EEG and EMG signal. By calculating the signal power values on two predetermined frequency bands (one covering only the range of the EEG and one covering the range of EEG and EMG) and generating a ratio that is compared to a threshold. If a high frequency EEG inducing drug was administered the device switches to “NMDA mode” instead of using the “normal mode” to determine the state index of the subject. The “normal mode” is disclosed in the U.S. Pat. No. 6,801,803 (entropy-based monitoring).

The patent application EP 1 563 789 A1 contains a method for monitoring the neurological state of a patient by obtaining a cortex-related biosignal and a subcortex-related biosignal.

At least two indicators will be used to calculate the state of the patient: the cortex-related frontal EEG and the subcortical activity of the patient based on the bioimpedance signal. The composite indicator at least consists of the EEG indicator and the skin conductive indicator. However the patent contains the possibility of an EMG indicator and an ECG indicator. The signal will be obtained by a set of four electrodes.

Neither of the two above patent applications relates any of the recorded biosignal with data from infused anaesthetics to define a hybrid index indicative of the level of consciousness.

The BIS is described in U.S. Pat. Nos. 4,907,597, 5,010,891, 5,320,109; and 5,458,117. The patents describe various combinations of time-domain and frequency-domain subparameters, including a higher order spectral subparameter, to form a single index (BIS) that correlates to the clinical assessment of the patient for example carried out by the OAAS. The BIS is manufactured by Covidien.

The U.S. Pat. No. 6,801,803, titled “Method and apparatus for determining the cerebral state of a patient with fast response” characterizes the Entropy method which is commercialised in module, not a standalone device, by the company General Electric (GE). The Entropy is applied to generate two indices, the state entropy (SE) and the response entropy (RE). The SE is based on the entropy of the frequencies from 0 to 32 Hz of the recorded signal while the RE is based on a wider interval, i.e. from 0 to 47 Hz. Besides the Entropy, claim 7 of this patent includes the Lempel-Zev complexity algorithm in as well.

The patient state analyzer (PSA) is described in U.S. Pat. No. 6,317,627. The PSA is using a number of subparameters, defined in tables 1, 2 and 3 of the patent. Included are different frequency bands such as delta, gamma, alpha and beta activity and ratios such as relative power which are merged together into an index using a discriminatory function.

BIS, Entropy, Patient State Index all suffer from contamination of the EEG by the EMG, these two are very difficult to separate because they have vast spectral overlap, approximately from 10 Hz to 35 Hz. The present invention benefits from prior knowledge of amount of infused drugs, hence a more precise estimate of the EMG activity can be carried out, hence correcting the EEG and the final level of consciousness index.

The patent application WO 2005/072792 A “System for adaptive drug delivery” characterizes a system for control of administration of anaesthetics and other drugs. That system applies online adjustment of the model parameters, while the novelty of the present system is the application of a neural network for close to real time update of the model parameters, which gives a more robust control loop.

The European patent application EP 1 742 155 A2 is related to the determination of the clinical state of the subject, where one application is the determination of the nociceptive or antinociceptive state of a subject. Nociception normally refers to pain, while antinociception refers to the blocking or gradual suppression of nociception in pain pathways at a subcortical level. An index of nociception is calculated by a weighted average of the recorded signal. The present invention is different because it includes information from infusion pumps and used other methods to combine the measured data than what is disclosed in EP 1 742 155 A2.

The U.S. Pat. No. 6,631,291 describes a closed loop method and apparatus for controlling the administration of a hypnotic drug to a patient. An EEG signal data complexity measure is used as the feedback signal in a control loop for an anesthetic delivery unit to control hypnotic drug administration to the patient in a such way that the desired hypnotic level of the patient is achieved. The control algorithm in said patent does not include the use of a neural network.

The US Patent 20020117176 “Anaesthesia Control System” and U.S. Pat. No. 6,934,579 describes a system for measuring Auditory Evoked Potentials and deriving an index used in a control algorithm, however online adjustment of the model parameters by a neural network is not claimed.

The US Patent 20060009733 “BIS Closed loop anesthetic delivery” applies the bispectral index as a depth of anaesthesia sensor in a the control system, but it also uses an automated response monitoring system, however it does not claim online adjustment of the model parameters.

### I

### INTRODUCTION TO THE INVENTION

The present invention is based on the hypothesis that if information from infused volume, integral and derivative of infused volume, plasma concentration, effect-site concentration of the anaesthetics and features extracted from the EEG are combined, then a much more precise description of the patient's depth of anaesthesia can be achieved. In particular in sedated patients the available indices of consciousness based on EEG show a high number of fluctuations not related to the patients level of consciousness. These fluctuations are in many cases thought to be due to influence from the EMG. Knowing how much remifentanil has been infused makes it easier to compensate the index of the level of consciousness.

A new concept for defining the effect site concentration of anaesthetics and analgesics is presented as well. Instead of using a traditional compartment model approach, where a first step is definition of a pharmacokinetic model and then a pharmacodynamic model, here a fuzzy reasoner combined with a Hopfield network is used. The Hopfield network ensures online estimation of the model parameters, this means that the model can be tailored to the individual patient. The model is updated online including the specific behaviour of the individual patient, according to the way the patient responds to the infused drugs, this approach has been chosen in order to reduce errors due to both inter and intra individual variation.

The effect site concentrations of the anaesthetic (C_{eA}) and the analgesic (C_{eB}) are calculated either by the Schnider and Minto model or by a proprietary ANFIS model; the ANFIS model takes more parameters into account, that is the age, bmi, sex, infused volume over time and the derivate of the infused volume in order to calculate the effect site concentration. In an enhanced embodiment the fenotype/genotype of sensitivity to opioids is included as well. This parameter provides additional precision in the assessment of the effect of the opioid. Significant interindividual differences in opioid sensitivity can hamper effective pain treatment and increase the risk for substance abuse. Hence this information provides a safer infusion system.

### DEFINITION OF THE ELC

In a preferred embodiment the ELC is calculated as a combination of features extracted from the EEG. The extracted features are betaratio, deltaratio and burst suppression rate (BSR). Other frequencies ratios can be calculated as well. These parameters are fed into a linear multiple regression or an Adaptive Neuro Fuzzy Inference System (ANFIS) which in the first place is trained in order to establish the model parameters.

### Interaction Surface.

The combined effect of the two infused drugs, anaesthetics and analgesics, can be visualized by defining an interaction surface. Traditionally, the interaction surface is estimated by a sigmoidal model however this limits the surface to certain shapes. In this invention a data driven approach such as an adaptive neuro fuzzy inference system (ANFIS) is used which allows a more flexible surface shape. A novelty is that the output of the ANFIS, i.e. the results on the z-axis on FIG. 2, is a scale from 0 to 100, which is directly comparable with the EEG monitors of the level of consciousness.

The interaction surface, between the hypnotic drug (for example propofol) and the analgesics (for example remifentanil) is shown on FIG. 2. Isoboles can be extracted and a confidence interval is defined (shown in red on FIG. 3) based on the individual variation of each patient. Confidence intervals are defined for both the ELC and the CELC, and those should have a minimum overlap otherwise a warning or alarm is released.

### Hopfield Neural Network for Online Estimation of Effect Site Concentrations

This section illustrates the application of Hopfield neural networks (HNNs) to the on-line identification of the interaction between propofol and remifentanil using their effect site concentrations (Ce) and the corresponding EEG measure of effect.

A Hopfield net is a recurrent artificial neural network invented by John Hopfield. Hopfield nets serve as content-addressable memory systems with binary threshold units. They are guaranteed to converge to a local minimum, but convergence to one of the stored patterns is not guaranteed.

The units in Hopfield nets are binary threshold units, i.e. the units only take on two different values for their states and the value is determined by whether or not the units' input exceeds their threshold. Hopfield nets can either have units that take on values of 1 or −1, or units that take on values of 1 or 0. So, the two possible definitions for unit i's activation, aᵢ, are:

$$\begin{matrix}{a}_{i} ← \{ \begin{matrix}1 & if {\sum }_{j}^{ } {w}_{ij} {s}_{j} > {θ}_{i} , \\ - 1 & otherwise .\end{matrix} & ( 1 ) \\ {a}_{i} ← \{ \begin{matrix}1 & if {\sum }_{j}^{ } {w}_{ij} {s}_{j} > {θ}_{i} , \\ 0 & otherwise .\end{matrix} & ( 2 )\end{matrix}$$

Where:
wij is the strength of the connection weight from unit j to unit i (the weight of the connection).
sj is the state of unit j.
θi is the threshold of unit i.

FIG. 5 (13) shows the connection of the neural network, in the example a Hopfield neural network is used. The network is trained to update the effect site concentrations of the anaesthetics and the analgesics. This update is carried out online based on the difference in the CELC.

### Combined Drugs and EEG Index

The novelty of the present method described in this patent is its ability to produce a combined drugs and EEG index of the Level of Consciousness (CELC) which is less influenced by the EMG than other existing methods, because it takes into account the amount of drugs administered to the patient. It is known that opioids, such as remifentanil, produces more EMG and hence interferes with the final index. In the present invention, the amount of remifentanil is known, therefore compensation for increased EMG can be made.

### Warning System Based on Difference Between Drugs Interaction Surface and Monitored EEG

The level of consciousness can be monitored by, on one hand, a proprietary index derived from the EEG. This index is calculated and updated in real-time by feature extraction of the EEG. The EEG index has a delay in the 1 to 30 s range and as such serves as a specific measurement of the state of the patient. On the other hand, the level of consciousness can also be estimated by a model taking into account the interaction between the hypnotics (for example propofol) and the analgesics (for example remifentanil). The two estimates of the level of consciousness should be within reasonable agreement. FIG. 3 shows an example of the behavior of the two estimates of the level of consciousness. The red curve is the estimate by the drug interaction, where the width of the curve corresponds to a confidence interval. The black curve is the estimate of the level of consciousness by EEG (ELC) where the width of the curve corresponds to the confidence interval of choice. The two curves should have a minimum overlap in a such way that there is not significant difference between the two. When the two estimates do not overlap, as is the case at time C in FIG. 3, a faulty situation could have occurred and a warning is given. In this case, where the ELC is higher than the level of consciousness estimated by drugs, the reason could be that the infusion device is not infusing the drugs correctly to the patient. This could be because the intravenous catheter line is not correctly attached to the patient. The opposite event could be that the ELC is much lower than the drug interaction value, this would be the case when the patient has a high sensitivity to the infused anaesthetics. In this case the effect site concentration will be updated the Hopfield neural network.

### FIGURE LEGENDS

FIG. 1. Overview of the complete invention including drugs interaction model and EEG recording for assessing precise levels of the level of consciousness during wake, sedation and general anaesthesia.

FIG. 2. The drug interaction surface.

FIG. 3. Expected level of consciousness (red curve, where the width is the confidence interval) according to the drugs interaction and by EEG (ELC, black curve, with corresponding confidence interval).

FIG. 4. Combination of infused drug concentrations and measured level of consciousness for defining a new index of the level of consciousness (CELC)

FIG. 5. Detailed description of the invention. The figure shows how a neural network can be added to the system in a such way that the parameters can be updated online.

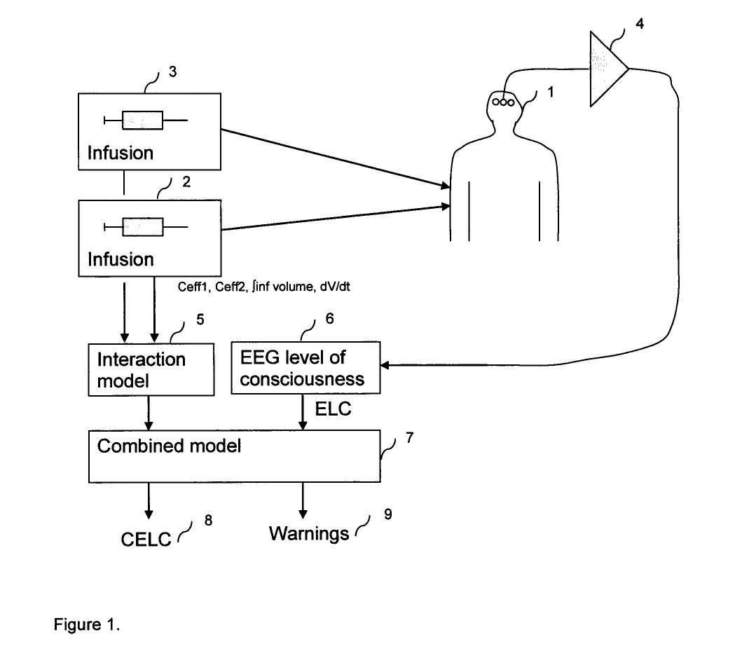

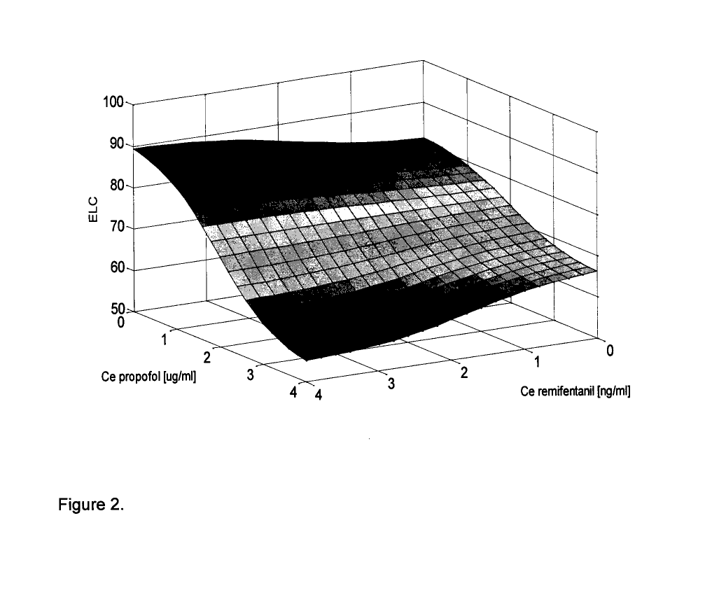

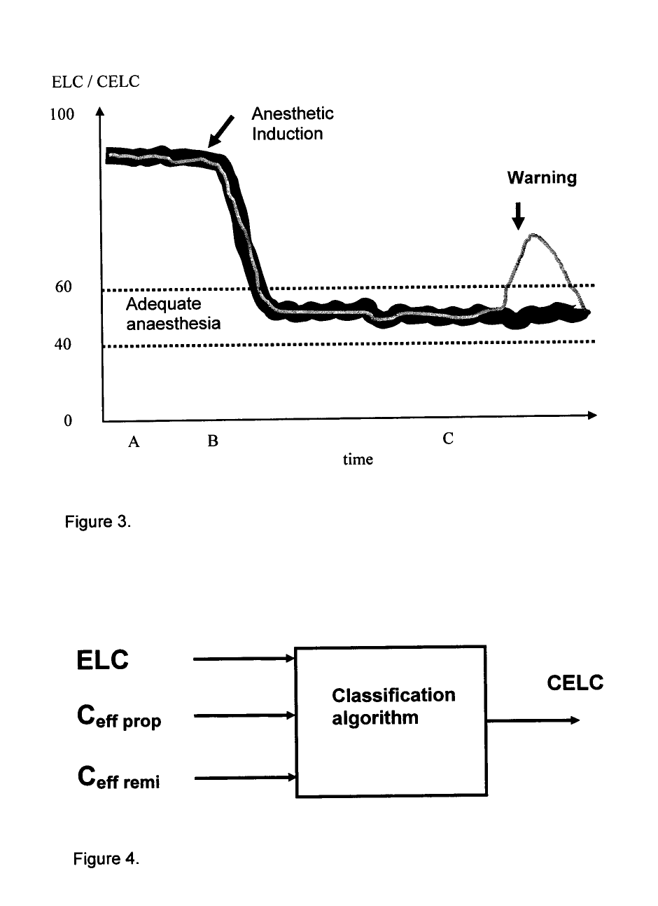

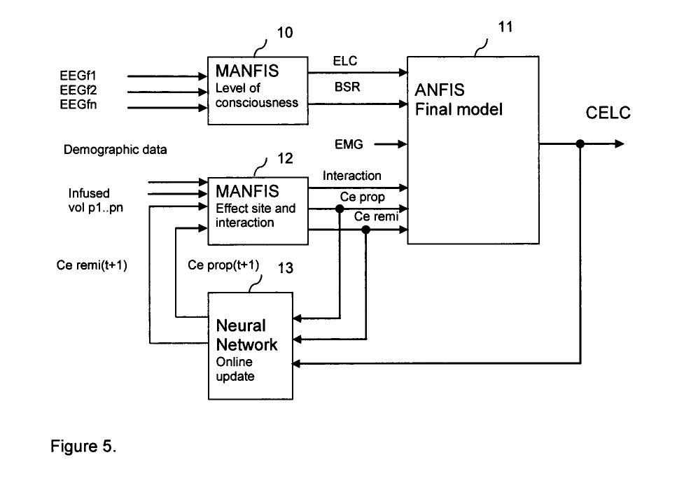

1. An apparatus for improving a precision of estimate of a level of consciousness of a patient during sedation or general anaesthesia; the apparatus comprising the following:
a) An EEG device for estimating a level of the consciousness (ELC);
b) one or more devices for delivering the anaesthetics and analgesics to the patient;
c) said device is characterised by a calculating module configured for performing the following steps:
i) determining in real time a model for relation between a drug dose and an effect of hypnotic and analgesic drugs administered to the patient; wherein said model is tailored to the patient; and
ii) calculating based on the model, effect of the hypnotic and the analgesic drugs and combining the effect with the ELC to establish an index of the level of consciousness (CELC).

2. The apparatus according to claim 1 characterized by sensors for monitoring the patients EEG and deriving an index of the level of consciousness (ELC) from features and solutions to the Schrodinger operator.

3. The apparatus according to claim 1 wherein the individual model characterised by the use of a neural network for defining the model.

4. The apparatus according to claim 1, further comprising a warning unit configured for activating an alarm if a difference between the ELC and the CELC is larger than a threshold; wherein the threshold is defined as a minimum overlap between two confidence intervals of ELC and CELC.

5. The apparatus according to claim 1 wherein the processor is further configured for verifying that a combination of hypnotic and analgesic drugs does not give raise to falsely high concentrations of the drug.

6. The apparatus according to claim 1, wherein the processor is further configured for defining the optimal path on the interaction surface; said interaction surface is defined by a data driven approach such as an Adaptive Neuro Fuzzy Inference System.

7. The apparatus of claim 2, wherein the features comprise spectral parameters.

8. The apparatus of claim 1, further comprising a display adapted for displaying the level of consciousness.

9. The apparatus of claim 6, wherein the hybrid fuzzy reasoner comprises an Adaptive Neuro Fuzzy Inference System (ANFIS).

10. The apparatus of claim 8, further comprising a table reflecting a response of the patient.

11. The apparatus of claim 1, wherein the calculating module is further configured for calculating over a period safety changes characterized by a difference between the ELC and the CELC.

12. The apparatus of claim 1, wherein the calculating module is further configured for calculating over a period safety considerations based on speed and changes in the infusion speed of the hypnotic and analgesic drugs.

13. The apparatus according to claim 1 wherein the calculating module is further configured for providing a variable delay of the ELC to the CELC.

14. The apparatus of claim 1, wherein the calculating module is further configured for establishing the optimal path on the interaction surface in case that more than one drug is administered.
